(19) United States
(12) Patent Application Publication
(10) Pub. No.: US 2016/0183793 A1
(43) Pub. Date: Jun. 30, 2016

(54) Wireless Catheter System for Cardiac Electrophysiology Study

(71) Applicants: Jamil MOGUL, Saratoga, CA (US); Anatoly RYZHIKH, Moscow (RU)

(72) Inventors: Jamil MOGUL, Saratoga, CA (US); Anatoly RYZHIKH, Moscow (RU)

(21) Appl. No.: 14/583,150

(22) Filed: Dec. 25, 2014

(51) Int. Cl. A61B 5/00; A61B 5/042

(52) U.S. Cl. CPC A61B 5/0006; A61B 5/0422; A61B 5/6852; A61B 5/4836; A61B 2560/045

(57) ABSTRACT

A Wireless Catheter Module (WCM) is adapted for attachment to a diagnostic catheter to enable the bio-electrical signals, analog signals picked up by the probing electrodes imbedded in the attached catheter, to be captured and digitized by A-to-D converter electronics in the WCM whereupon packets of the digitized signals can then be transmitted wirelessly from the WCM to a remote Wireless Base Station (WBS) forming a receiver component of the present system. The WBS is adapted for operative connected to an EP recording and cardiac stimulation system. In cases where multiple catheters are to be used in a study, each catheter may be fitted with an appropriately configured WCM and the catheter modules can communicate simultaneously with the WBS.

### BACKGROUND

1. Field of the Invention

The present invention relates generally to cardiac electrophysiology apparatus and more particularly to a wireless catheter system for use in conducting cardiac electrophysiology studies.

2. Prior Art

A human heart has six main internal regions or areas including the sinus, right atrium, left atrium, right ventricle, left ventricle, and the His-Purkinje system. A junction of two of these regions or areas is known as a node.

The His-Purkinje system is a bundle or collection of heart muscle cells specialized for electrical conduction that transmits the electrical impulses from the AV node (located between the atria and the ventricles) to the point of the apex of the fascicular branches via the bundle branches. The fascicular branches then lead to the Purkinje fibers, which provide electrical conduction to the ventricles, causing the cardiac muscle of the ventricles to contract at a paced interval.

The cardiac cells of the sinoatrial (SA) and the atrioventricular (AV) nodes, and the His-Purkinje system act like both muscle and nervous tissues and are capable of initiating an electrical activity, and both have a characteristic of automaticity—an occurrence of spontaneous, repeated cellular depolarization that results in an action potential (i.e., voltage) or bio-electrical signals.

An electrical impulse originates from the SA node (the natural pacemaker) which has the fastest intrinsic automaticity rate that determines the heart rate. The impulse stimulates the contiguous cardiac cells and causes cardiac depolarization via the conduction pathways. Cardiac repolarization occurs when the cells return to their original resting state.

In addition to the SA node, the AV node and the His-Purkinje system also have the ability to function as redundant pacemakers whose automaticity rate is slower than that of the SA node. Cardiac depolarization from an impulse of the SA node suppresses these redundant pacemakers. If the SA node fails to maintain its pacemaking activity, the AV node can allow a continuous cardiac electrical activity. The His-Purkinje system serves as a back-up subsidiary pacemaker in case the SA node or AV node conduction fails.

The electrical activity of the redundant pacemakers (AV node and Perkinje fibers) that naturally have lower rates than the rate of the dominant pacemaker (SA node) is acknowledged only by the cardiac physiological system when the SA node rate falls below those of the redundant pacemakers. As such, the electrical activity of the redundant pacemakers which emerge to sustain a heart rate when the dominant pacemaker fails is called an escape mechanism, which provides a slower rate. In other words, the redundant pacemakers (that emerge when the dominant pacemaker fails) result in a lower heart rate, and their electrical activity is called an escape mechanism.

The SA nodal impulse then depolarizes the anterior, middle and posterior internodal tracts; the AV node; the His bundle; the right and left bundle branches; the anterior-superior and posterior-inferior divisions of the left bundle and the Purkinje fibers.

Normally, Sinus rhythm of a heart occurs when the SA node spontaneously and repeatedly generates an electrical impulse which, in a waveform, propagates through the cardiac atria and ventricles and depolarizes the myocardial structure by following the natural, specialized conduction pathways in the heart. The parasympathetic system normally slows the automaticity rate of the SA node from 100 beats per minutes to about 70 beats per minutes (bpm). The limits (the normal range) of the rate of the rhythm are 50 bpm and 120 bpm.

Cardiac arrhythmia, as a disease, occurs when the heart rate is persistently out of the normal range; faster than the normal heart rate is termed tachycardia, while slower than the normal heart rate is termed bradycardia. Also, disturbances in the rhythm are termed sinus arrhythmia.

The sinus arrhythmia includes conduction blocks, delays and escapes, and premature and fusion beats. The other cardiac arrhythmias are supraventricular tachycardia, atrial flutter, atrial fibrillation, Wolf-Parkinson-White syndrome, accelerated idioventricular rhythm, ventricular tachycardia, Torsades de Pointes (polymorphic ventricular tachycardia), ventricular fibrillation and asystol.

To clinically diagnose a cardiac arrhythmia, an electrophysiology (EP) study is conducted by using multiple diagnostic EP catheters in the catheter laboratory of a hospital. Examples of steerable diagnostic EP catheters are disclosed in U.S. patents to Jamil Mogul numbers U.S. Pat. Nos. 6,829,497, 7,122,020 and 7,269,452, the disclosures of which are expressly incorporated herein by reference.

A diagnostic EP catheter is mainly made of tubing, called catheter shaft, comprising a portion called the distal shaft and another portion called the proximal shaft, which portions are bonded together. The distal shaft externally contains a tip electrode and has multiple ring electrodes. The distal shaft is pre-curved into a fixed-curve shape, or is deflectable or steerable via a steering mechanism. Both the distal shaft and the proximal shaft internally contain electrical wires for the electrodes and mechanical wires for the steering mechanism, and also contain other parts to provide optimal electrical/mechanical functionality. The proximal shaft is attached to a connector for the electrodes in the case of a fixed-curve distal shaft, or to a handle that contains the steering mechanism and the connector in the case of a deflectable distal shaft.

The EP study is a minimally invasive diagnostic procedure which is performed percutaneously as follows: (a) punctures are made into the groin and/or neck; (b) introducer sheaths are inserted into the femoral vein; (c) multiple diagnostic EP catheters of a fixed-curve (non-steerable) or steerable distal shaft are inserted into the sheaths and, under fluoroscopic guidance, are advanced through the blood vasculature, and placed into a desired intracardiac region of the heart such as the right atrium, the His bundle region, and/or the right ventricle; and (d) once in position, the EP catheters are connected to electrogram-recording and cardiac stimulation (pacing) equipment which in turn is attached to a monitor that displays the intracardiac electrograms obtained from the electrodes of the catheters.

During the catheter insertion, the designed axial rigidity (column strength) and lateral flexure of the catheter shaft provide adequate pushability and trackability for traversing the catheter shaft through tortuous vasculature so as to access the interior of the heart. A particular shape of fixed-curve distal shaft which is formed for a certain intracardiac region aids in accessing the region and placing the distal shaft into that region while making contact with a targeted endocardium. The distal shaft is maneuvered via twisting (torqueing), moving backward or forward, or steering to position and/or to place the distal shaft's electrodes in intimate contact with the targeted tissue sites for sensing bio-electric signals (electrograms), and/or stimulating the endocardium.

During an EP study procedure, right atrial pacing and recording, His bundle recording, and right ventricular pacing and recording are often also performed. The catheters are repositioned numerous times, and pacing and recording are done at various areas within the heart. If an arrhythmia is induced via cardiac stimulation, it may be terminated by rapidly pacing the heart or by defibrillation or cardioversion.

Once all pacing and recording is completed during the EP study, the catheters are withdrawn and the introducer sheaths are removed. The EP physician documents the procedure and the results of the study along with any recommendations for treatment.

### The Problems Addressed

As can be noted from the above, the use of multiple EP catheters (often four to five) requires the use of multiple interconnecting multi-conductor or single-pin cables during the EP study. The interconnecting cables often become intrusive or entangled and/or restrict to a certain degree the EP physician's movement and/or the handling and maneuverability of the catheters. Additionally, the cables, which are often re-sterilizable and reusable, tend to break or get damaged during repeated interconnections at a first use or after a reuse. Moreover, multiple interconnecting cables and their handling, re-sterilization and storage are a cost factor for an EP study, and the replacement of damaged cables obviously increases the cost.

### A Solution to the Problems

To address the issues mentioned above, a wireless catheter system having two principal parts: a Wireless Catheter Module (WCM) for attachment to a diagnostic EP catheter, and a corresponding Wireless Base Station (WBS) for wireless connection to a remotely positioned EP recording and cardiac stimulation system has been developed.

### BRIEF STATEMENT OF THE INVENTION

In accordance with the present invention, a Wireless Catheter Module (WCM) is adapted for attachment to the handle of a steerable or non-steerable diagnostic catheter to enable the bio-electrical signals (electrograms), analog signals (in millivolt DC) picked up by the probing electrodes imbedded in an attached catheter, to be captured and digitized by A-to-D converter electronics in the WCM whereupon packets of the digitized signals can then be transmitted wirelessly from the WCM to a remote Wireless Base Station (WBS) forming a receiver component of the present system. The WBS is adapted for operative connected to an EP recording and cardiac stimulation system. In cases where multiple catheters are to be used in a study, each catheter may be fitted with an appropriately configured WCM and the catheter modules can communicate simultaneously with the WBS.

An important advantage of the present invention is that it eliminates the need for a diagnostic catheter to be tethered to an EP recording and cardiac stimulation system.

Another advantage of the present invention is that it allows a plurality of diagnostic catheters to simultaneously communicate with an EP recording and cardiac stimulation system without having to be connected thereto with a plurality of data cables.

Still another advantage of the present invention is that it avoids the likelihood of catheter damage, difficulty of patient access, and signal connection error presently resulting from the use of wired connection of multiple catheters to an EP recording and cardiac stimulation system.

These and other advantages of the present invention will become apparent to those skilled in the art after a reading of the following detailed disclosure of embodiments of the present invention.

### IN THE DRAWING

FIGS. 1A and 1B are generalized diagrams respectively illustrating a wireless catheter module (WCM) attached to a diagnostic EP catheter, and a paired remote wireless base station (WBS), the several units forming a wireless catheter system in accordance with the present invention;

FIG. 2 depicts the butt end of a catheter fitted with a multi-pin electro-mechanical connector component for mating with the connector shown in FIGS. 3A and 3B;

FIG. 3A illustrates the principal components of the wireless catheter module (WCM) and its a multi-pin electro-mechanical connector component as shown in FIG. 1A;

FIG. 3B is an end view showing details of the a multi-pin electro-mechanical connector component of FIG. 3A;

FIG. 4 schematically illustrates the principal components of the remote wireless base station (WBS) shown in FIG. 1A;

FIG. 5 is a block diagram schematically illustrating the operative electrical circuits and components of the wireless catheter module (WCM) shown in FIGS. 1A and 3A; and

FIG. 6 is a block diagram schematically illustrating the operative electrical circuits and components of the wireless base station (WBS) shown in FIGS. 1B and 4.

### DETAILED DESCRIPTION OF THE EMBODIMENTS

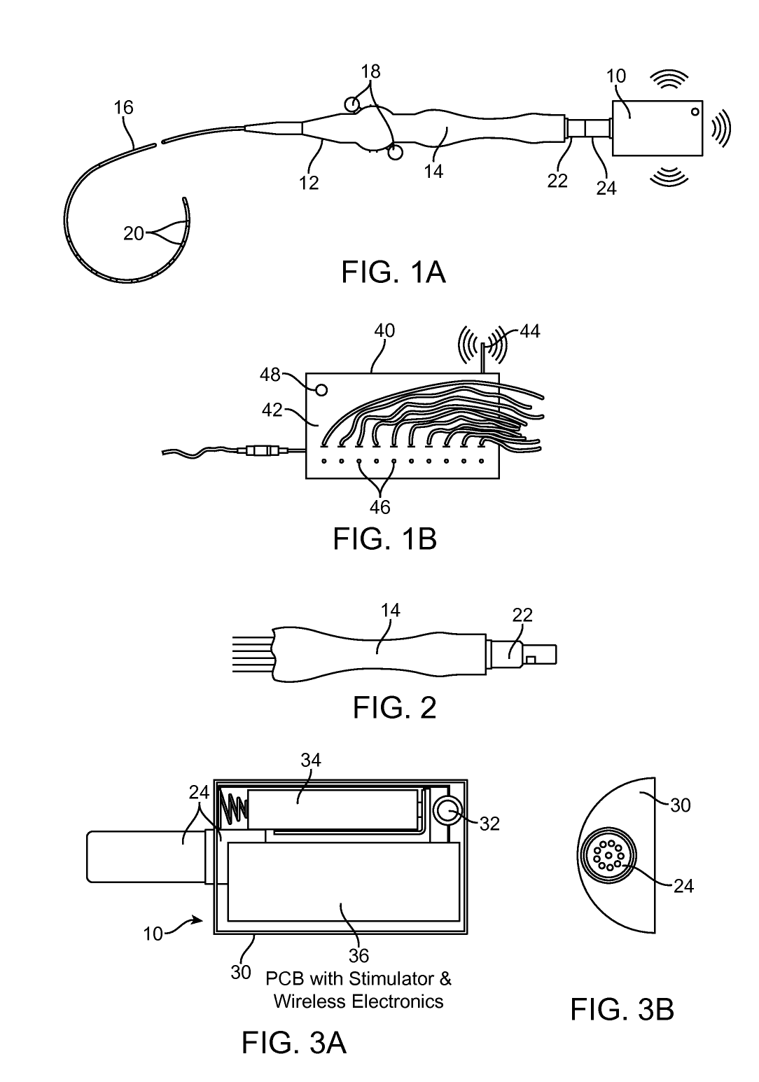

Referring now to the Drawing wherein an embodiment of a wireless catheter system in accordance with the present invention is generally depicted in FIGS. 1A and 1B. The system includes a wireless catheter module (WCM) 10 attached to a modified diagnostic EP catheter device 12, and a paired remote wireless base station (WBS) 16.

As is well known from prior art including the above mentioned US patents, a typical prior art catheter apparatus usually includes a handle such as is shown at 14 in FIG. 1 and a catheter body 16 extending from one end of the handle. The catheter may be steerable or non-steerable. In the case of the steerable catheter the handle is usually formed by mating shells joined together to form an enclosure for containing a catheter steering mechanism that might include slidable or pivotable levers 18 that extend outside of the handle to permit manipulation of the catheter body by a user.

The catheter body 16 is usually comprised of a length of flexible tubing having conductive tip electrode and a plurality of ring or otherwise configured electrodes 20 positioned at intervals along a distal end portion of the catheter body. The proximal end of the catheter body is attached to the front end of the handle. Extending internally through and along the length of the catheter body are one or more steering wires (not shown) having the distal ends thereof affixed to the distal end of the catheter body, and the proximal ends thereof connected to the levers 18 to permit steering deflection of the catheter body.

Also extending internally through and along the length of the catheter body are a plurality of electrical signal wires (not shown) having their distal ends connected to corresponding ones of the electrodes 20. In the prior art devices, the proximal ends of the wires extend out of the proximal end of the catheter body and through the handle 13 to terminate in a cable connector to which a cable leading to an EP recording and cardiac stimulation system or other local signal processing unit is connected.

However, in accordance with the present invention the butt end of the handle is modified to include a special electro-mechanical connector part 22 (see also FIG. 2) adapted to mate with a corresponding multi-pin connector part 24. The connector parts 22-24 serve the dual function of electrically connecting the catheter signal wires to the WCM and at the same time physically connecting the WCM 10 to the steerable catheter 12 to form a unitary wireless catheter unit.

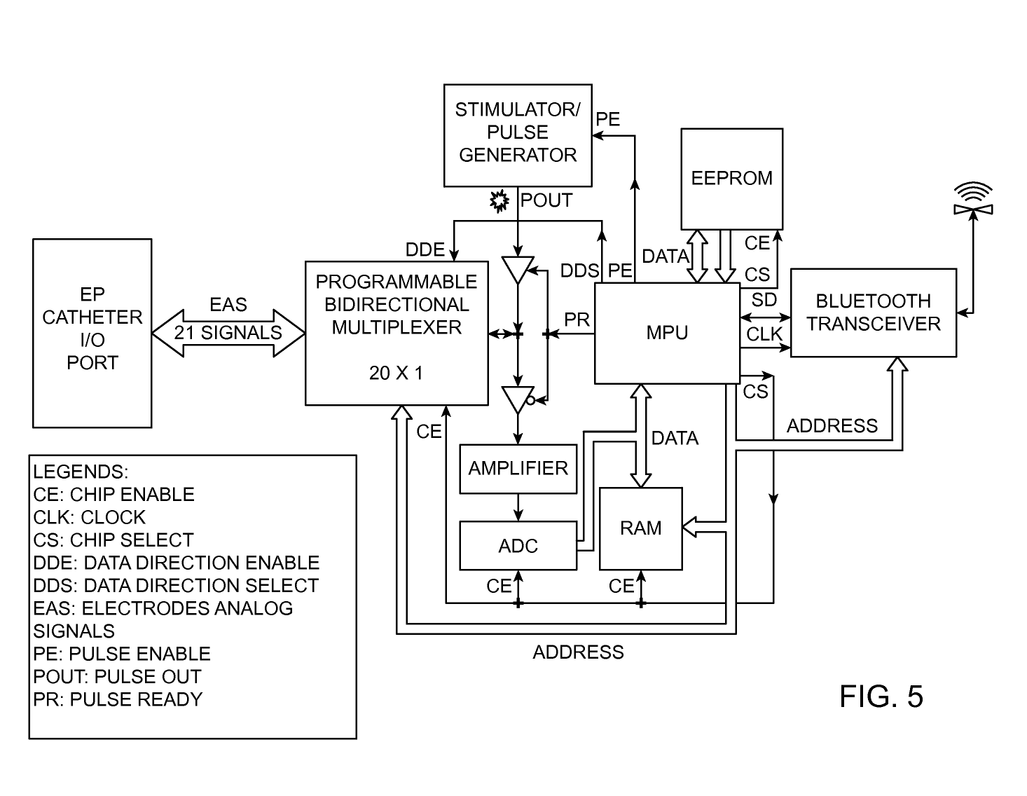

Referring now additionally to FIG. 3, the WCM 10 consists of a sealed enclosure 30 that externally includes the multi-pin connector 24 (see also FIG. 3B) and a power on/off LED switch 32. Within the enclosure is a battery 34 and interconnect wires (not shown) for connecting a printed circuit board (PCB) 36 to the connector 24. Mounted upon the PCB are (not shown) a programming chip, a power supply and transceiver wireless electronics, analog (A)-to-digital (D) converter electronics, and cardiac stimulator electronics all of which are further discussed below with respect to the circuit diagram of FIG. 5. The multi-pin connector component 24 is for interfacing the WCM to a mating diagnostic EP catheter connector component as discussed above.

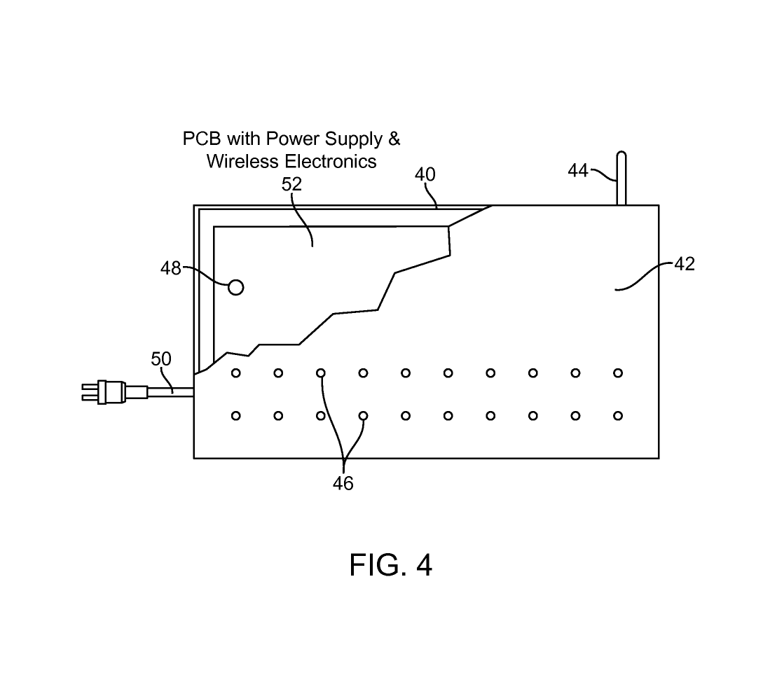

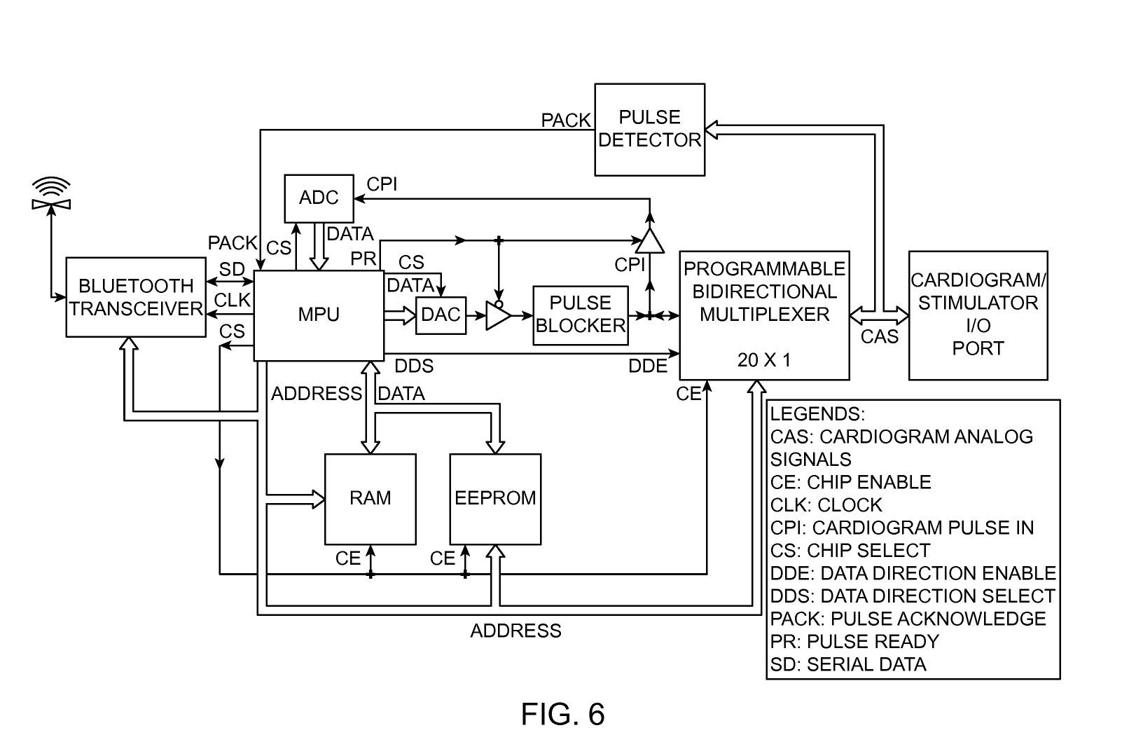

Returning to FIG. 1B and further to FIG. 4, the WBS 40 includes an enclosure 42 that externally carries an antenna 44, multiple single-pin receptacle connectors 46, a power on/off switch 48, and a power cord 50. The WBS internally contains interconnect wires (not shown) for the connectors 46, and a PCB 52. Mounted on the PCB (and reflected in the circuit diagram of FIG. 6) are a power supply and transceiver wireless electronics, and D-to-A and A-to-D converter electronics. The receptacle connectors function as multiple channels representing the number and order of the electrodes of the catheter.

During use, a diagnostic EP catheter with multiple electrodes will be connected to the WCM, and the power switch will be pressed to the on-position in order to supply power to the unit. The WBS, via the multiple channels 46, will be interconnected to an EP recording and cardiac stimulation system. The power cord will be connected to an electrical outlet, and the power switch will be turned on.

During an EP study, when the catheter electrodes are in contact with the targeted endocardium, a bio-electrical signal (electrogram), which is an analog signal (in millivolt DC), will be picked up by each catheter electrode and sent via the wires imbedded in the catheter to the connected WCM. The analog signals will be captured and digitized by the A-to-D converter electronics of the WCM, and Packets of the digitized signals will be sent wirelessly by the WCM to the remote WBS.

The digitized wireless signals received by the WBS will then be converted back to analog signals by the D-to-A converter in the WBS electronics. The converted analog signals will be captured by the interconnected EP recording and cardiac stimulation system which will record and display the analog signal in millivolts on its attached monitor.

To stimulate a targeted endocardium, the WBS will receive, via an identifiable channel, a DC voltage of a certain value from the interconnected EP recording and cardiac stimulation system. The DC voltage value can vary from 5 volts to 20 volts or more. The certain value of the DC volts will be digitized via the A-to-D converter and assigned a distinct signal to indicate the certain voltage value and the identification of the channel that corresponds to the electrode number. The digitized packet of the distinct signal will then be wirelessly transmitted back to the WCM.

Following receipt of the distinct signal (for the certain DC volts and the electrode number) by the WCM, the programming chip in the WCM will process the signal to translate the signal into the certain DC voltage value and identify the electrode number for cardiac stimulation. The processed information will then be communicated to the stimulator electronics which will activate the stimulator to send the certain DC volts to the intended catheter electrode so as to stimulate the targeted endocardium that is in contact with the electrode.

FIG. 5 is a block diagram schematically illustrating the operative electrical circuits and components of the wireless catheter module (WCM) shown in FIGS. 1A and 3A. The circuit diagram is believed to be self-explanatory and well within the realm of understanding of those skilled in the relevant art.

FIG. 6 is a block diagram schematically illustrating the operative electrical circuits and components of the wireless base station (WBS) shown in FIGS. 1B and 4. The circuit diagram is believed to be self-explanatory and well within the realm of understanding of those skilled in the relevant art.

Although the present invention has been described above in terms of a specific embodiment, and various applications have been suggested, it is anticipated that after reading the foregoing disclosure, numerous other embodiments and applications of the present invention will become apparent to those skilled in the art. It is therefore intended that this disclosure be considered as exemplary rather than limiting, and that the following claims be interpreted as covering all alternatives, modifications and embodiments as fall within the true spirit and scope of the invention.

1. A wireless catheter data communication system, comprising:
a wireless catheter module (WCM) for attachment to a diagnostic EP catheter and operative to capture analog bio-electrical signals picked up by probing electrodes imbedded in the catheter, to digitize the captured signals, to packetize the digitized signals, and to wirelessly transmit the packetized signals; and
a wireless base station (WBS) for remotely receiving the transmitted packetized signals and operative to convert the packetized signals back to analog signals for input to an EP recording and cardiac stimulation system.

2. A wireless catheter data communication system as recited in claim 1 wherein the identity of the probing electrode corresponding to each captured signal is also determined and forms an identifying part of each packetized and transmitted signal.

3. A wireless catheter data communication system as recited in claim 2 wherein said a wireless base station (WBS) is operative to receive stimulating signals from the EP recording and cardiac stimulation system for stimulating a targeted endocardium, to digitize the stimulating signals, to packetize the digitized stimulating signals, and to wirelessly transmit the packetized stimulating signals and the identity of the catheter electrode to be used to stimulate the targeted endocardium back to the wireless catheter module for conversion to a specific voltage value for application to the identified electrode to stimulate the targeted endocardium.

4. A wireless catheter data communication system as recited in claim 1 and further comprising an electro-mechanical connector for physically connecting the wireless catheter module to a diagnostic EP catheter, and for electrically connecting each electrode of the catheter to a corresponding signal processing channel of the wireless catheter module.

5. A wireless catheter data communication system as recited in claim 4 wherein the electro-mechanical connector includes a first part for attachment to the butt end of the handle of a diagnostic EP catheter, and a mating second part for connection to the wireless catheter module.

6. A wireless catheter data communication system as recited in claim 1 wherein the wireless base station is capable of simultaneously receiving and processing transmitted packetized signals from multiple wireless catheter modules for input to an EP recording and cardiac stimulation system.

7. A wireless catheter probe and data communication system comprising:
a diagnostic EP catheter including an elongated catheter body including a plurality of probing electrodes disposed along the distal end portion thereof, a handle member affixed to the proximal end of the catheter body, and one part of a two part electro-mechanical multi-pin connector affixed to the handle a plurality of signal wires disposed internally of the catheter body and extending along the length thereof with one end of each wire being connected to one of the electrodes and the other end of each wire extending into the handle and being connected to one pin of the multi-pin connector;
a wireless catheter module (WCM) attached to the catheter by the second part of the two part multi-pin connector, the wireless catheter module being operative to capture analog bio-electrical signals picked up by the catheter electrodes and communicated thereto along the wires and through the multi-pin connector, to digitize the captured signals, to packetize the digitized signals, and to wirelessly transmit the packetized signals; and
a wireless base station (WBS) for remotely receiving the transmitted packetized signals and operative to convert the packetized signals back to analog signals for input to an EP recording and cardiac stimulation system.

8. A wireless catheter probe and data communication system as recited in claim 7 wherein the identity of the probing electrode corresponding to each captured signal is also determined and forms an identifying part of each packetized and transmitted signal.

9. A wireless catheter probe and data communication system as recited in claim 8 wherein the wireless base station (WBS) is operative to receive stimulating signals from the EP recording and cardiac stimulation system for stimulating a targeted endocardium, to digitize the stimulating signals, to packetize the digitized stimulating signals, and to wirelessly transmit the packetized stimulating signals and the identity of the catheter electrode to be used to stimulate the targeted endocardium back to the wireless catheter module for conversion to a specific voltage value for application to the identified electrode to stimulate the targeted endocardium.

10. A wireless catheter probe and data communication system as recited in claim 7 wherein the wireless base station is capable of simultaneously receiving and processing transmitted packetized signals from multiple wireless catheter modules for input to an EP recording and cardiac stimulation system.
